(19) United States
(12) Patent Application Publication
(10) Pub. No.: US 2020/0017916 A1
(43) Pub. Date: Jan. 16, 2020

(54) BLOOD-BASED BIOMARKERS FOR THE DETECTION OF COLORECTAL CANCER

(71) Applicant: WRIGHT STATE UNIVERSITY, Dayton, OH (US)

(72) Inventor: Hongmei Ren, Xenia, OH (US)

(21) Appl. No.: 16/428,517

(22) Filed: May 31, 2019

Related U.S. Application Data
(60) Provisional application No. 62/678,655, filed on May 31, 2018.

(51) Int. Cl. C12Q 1/6886

(52) U.S. Cl. CPC C12Q 1/6886; C12Q 2600/112; C12Q 2600/154

(57) ABSTRACT

A method using genome-wide methylation profiling to investigate DNA methylation alterations in peripheral blood t for the diagnosis, prognosis, and/or prediction of therapy outcome of colorectal cancer (CRC). Use of accurate non-invasive biomarkers may be used to facilitate the early diagnosis of CRC.

### CROSS-REFERENCE TO RELATED APPLICATIONS

This application claims the benefit of U.S. Provisional Patent Application Ser. No. 62/678,655 entitled “BLOOD-BASED BIOMARKERS FOR THE DETECTION OF COLORECTAL CANCER” filed on May 31, 2018, the entirety of which is incorporated by reference herein.

### TECHNICAL FIELD

The innovation relates to the identification and use of biomarkers for use in clinic for predicting cancer risk.

### BACKGROUND

Colorectal cancer (CRC) is the third most common cancer diagnosed in both men and women and is the second leading cause of cancer-related death in the United States. The early detection of CRC significantly improves the prognosis of patients and is a key factor to reduce the mortality rate from CRC. It can be easily cured by surgical procedures if the cancer is diagnosed early, specifically before metastasis is established. The 5-year relative survival rate for early-stage CRC is 90%; for advanced stage IV CRC, the survival rate drops to about 5. However, only about 4 out of 10 CRC patients are diagnosed at the early stage, partially due to poor patient acceptance and/or sensitivity of available screening modalities. In comparison to colonoscopy, a blood-based test is non-invasive, convenient, and cost-effective with high acceptance by individuals, leading to greater screening compliance in the general population and a reduction in the incidence and mortality rates of this disease.

There is evidence that the risk of CRC can be modified by diet, lifestyle and environmental factors, which suggests epigenetic mechanisms are associated with CRC initiation and progression. Epigenetic mechanisms are heritable chemical modifications of DNA and chromatin involving alterations in DNA methylation, histone modifications and small noncoding microRNAs (miRNAs), which induce chromatin structural changes, thereby affecting gene activity. DNA methylation represents a more stable source of biological information than RNA or the expression of most proteins. It is the most common modification in the mammalian genome and occurs when a methyl group is added onto the C5 position of cytosine, thereby modifying gene function and affecting gene expression. Most DNA methylation occurs at cytosine residues that precede guanine residues, or CpG dinucleotides, which tend to cluster in DNA domains known as CpG islands.

The relationship between methylation and gene expression is complex. In general, DNA methylation of gene promoters is associated with transcriptional silencing, whereas methylation in gene bodies is associated with increased gene expression. Strong correlations between gene expression and CpG islands and island shores were demonstrated. Global hypomethylation is thought to influence CRC development by inducing chromosomal instability.

DNA methylation patterns in peripheral blood can be informative noninvasive biomarkers of cancer risk and prognosis with a high sensitivity and specificity. DNA methylation pattern alterations in the blood cells may reflect the microenvironment components which support cancer initiation and methylome changes in blood may also reflect changes that occur in colon cells during CRC progression.

In previous studies, a variety of epigenetic biomarkers have been evaluated in colorectal cancer for early detection and prognosis prediction, however, most of the studies focused on a single gene. For example, SEPT9 showed abnormal hypomethylation at its promoters and was considered to be a biomarker for CRC cancer detection. However, the sensitivity of SEPT9 is 48.2% for CRC stages I-IV, but much lower (11.2%) for the precancerous condition, advanced adenoma.

In recent years, genome-wide methylation profiling can help us understand the molecular mechanisms involved in CRC initiation and progression. There is a need for a less-invasive and accurate test for detecting CRC, especially in the early stage of the disease.

### SUMMARY

The following presents a simplified summary of the innovation in order to provide a basic understanding of some aspects of the innovation. This summary is not an extensive overview of the innovation. It is not intended to identify key/critical elements of the innovation or to delineate the scope of the innovation. Its sole purpose is to present some concepts of the innovation in a simplified form as a prelude to the more detailed description that is presented later.

Most previous studies on blood-based DNA methylation biomarkers have relied on testing a limited number of pre-selected genes and on the use of non-quantitative detection methods, such as gel-based methylation-specific PCR.

A method according to the innovation can include genome-wide methylation profiling to investigate DNA methylation alterations in peripheral blood on colorectal cancer (CRC) initiation and progression. In particular, the method employs accurate non-invasive biomarkers to facilitate the early diagnosis of CRC. In one embodiment, this may be addressed in a comprehensive fashion by identifying DNA methylation alterations during CRC progression and development in blood and tissue specimens, and integrates with gene transcriptional changes.

In one embodiment, a bisulfite sequencing method can be performed to identify differential methylated regions (DMRs) in peripheral blood samples for a CRC patient versus a control group. The bisulfite sequencing analysis can result in the identification of a plurality of alterations in the methylome landscape in peripheral blood of patients with CRC.

The results are that differentially methylated regions (DMRs) associated with the gene body regions of Ras-related genes are hypermethylated. The activation of Ras signaling is involved in CRC initiation and development. Because methylation in gene bodies is generally associated with increased gene expression, it can reasonably be deduced that DNA methylation alterations in peripheral blood contribute to the activation of Ras signaling and colorectal tumorigenesis. In contrast, most DMRs associated with genebody regions of Rac-related genes were hypomethylated, suggesting DNA methylation alterations may inhibit Rac signaling, which is an important regulator of Arp2/3-dependent actin polymerization and phagocytosis of invading pathogens. DNA methylation alterations can be in the endocytosis pathway. One important innate immune defense mechanism is the ingestion of extracellular macromolecules through endocytosis or phagocytosis of whole bacteria in order to remove the inflammatory stimuli. DNA methylation alterations in CRC greatly compromise the ability of intestinal epithelial cells to respond to invading pathogens.

A genome-wide methylation analysis was conducted in CRC tumors (N=10) compared to adjacent normal tissues (N=10) to reveal functional genes with significant aberrant DNA methylation during carcinogenesis. The age and gender of tissue donors were comparable to blood donors.

Integrated analysis between the transcriptome and methylation profile in CRC were performed to reveal the DNA methylation changes in both CRC peripheral blood and tumors, and the underlying regulatory mechanisms of the impact of DNA methylation alteration on gene expression during CRC development. Genes with overlapping DMRs in blood and tumors and altered gene expression were selected as potential candidate biomarkers. Correlation analysis on hypermethylated or hypomethylated overlapping DMRs in both tumor tissue and blood using integrated reduced representation bisulfite sequencing (RRBS) and RNA-Seq analysis was then performed.

According to an aspect, the innovation provides a method for identifying cancer-related DNA methylation alterations in peripheral blood. In one embodiment, genome-wide DNA methylation analysis may be performed to identify cancer-related DNA methylation alterations. In one embodiment, the DNA methylation alterations may be used to identify CRC. In one embodiment, the DNA methylation alteration may be used to diagnose the stage (e.g., early vs. late stage) of the CRC. In one embodiment, DNA methylation alterations in the Ras/Rac signaling pathway may be used t as blood-based diagnostic markers.

In one embodiment, the integrated analysis method according to the innovation allows for the efficient mapping of tumor-specific DNA methylation alterations in whole blood and tumor with an accompanying gene expression change, and screened a list of 96 genes associated with aberrant DMRs (shown in Table 3). For example, some of the genes including (MAPK9, RXRA, NR4A1, VAV2, ARHGEF4 and CELSR3) exhibit significant accuracy for the detection of CRC (e.g., AUC of CRC vs. control=1(>92% CI: 1, 1) for all DMRs). Some of genes (MAPK9, LRP1, ARAP1, COL4A2 and ARPC1B) can discriminate patients from early-stage to late-stage cancer in the peripheral blood DNA (e.g., AUC of late stage vs. early stage=0.884 (95% CI: 0.675, 1), 0.859 (95% CI: 0.612, 1), 0.848 (95% CI: 0.628, 1), 0.807 (95% CI: 0.571, 0.987) and 0.798 (95% CI: 0.55, 0.995), respectively).

To the accomplishment of the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1A is a bar graph of the number of DMRs between CRC vs. Control by gene subregions.

FIG. 1B is a bar graph of the number of DMRs between CRC vs. CpG islands.

FIG. 2 is a summary of the top 10 KEGG pathways and top 10 gene ontology-biological processes enriched for involved genes with DMRs.

FIG. 3 is a diagram depicting gene networks constructed from the most significant DMR-associated genes (red dots/red squares) and the involved top KEGG pathways (blue dots) and GO biological processes (green dots). Red dots represent a selected MIR for the detection of CRC, while pink squares represent a selected DMR for the discrimination of cancer clinical stages. The darkness of red is correlated with—log 10 transformed p values of cancer vs. control and dark red indicates a smaller p value. The size of the blue dots/green dots is correlated with—log 10 transformed enrichment p-values of KEGG pathways/GO biological processes and a large dot indicates a smaller p value.

FIG. 4A illustrates DMRs identified from CRC tumor tissue compared to surrounding normal tissues, and from peripheral blood in patients with CRC vs. healthy volunteers and is a Venn-diagram of DMRs generated from CRC tumor vs. normal tissue and peripheral blood in CRC vs. healthy controls.

FIG. 4B illustrates DMRs identified from CRC tumor tissue compared to surrounding normal tissues, and from peripheral blood in patients with CRC vs. healthy volunteers and is a scatter plot displaying Correlations between overlapping hyper-/hypomethylated DMRs in CRC tumor and peripheral blood.

FIG. 5A illustrates overlapping DMR located in promoter region of ARHGEF4 which is hypermethylated in both blood and cancer tissue, acting to suppress its gene expression wherein the drawing depicts representative methylation levels of the DMR in blood and cancer tissue.

FIG. 5B. illustrates overlapping DMR located in promoter region of ARHGEF4 which is hypermethylated in both blood and cancer tissue, acting to suppress its gene expression wherein the drawing depicts representative RNA-seq read coverage data from cancer tissue. ARHGEF4 is on the reverse strand.

FIG. 6A illustrates a graph depicting ROC curves and their associated areas under the curve (AUC) of selected DMRs for the detection of CRC (black curve) or the discrimination of cancer clinical stages (blue curve).

FIG. 6B illustrates a graph depicting ROC curves and their associated areas under the curve (AUC) of selected. DMRs for the detection of CRC (black curve) or the discrimination of cancer clinical stages (blue curve).

FIG. 6C illustrates a graph depicting ROC curves and their associated areas under the curve (AUC) of selected DMRs for the detection of CRC (black curve) or the discrimination of cancer clinical stages (blue curve).

FIG. 6D illustrates a graph depicting ROC curves and their associated areas under the curve (AUC) of selected DMRs for the detection of CRC (black curve) or the discrimination of cancer clinical stages (blue curve).

FIG. 6E illustrates a graph depicting ROC curves and their associated areas under the curve (AUC) of selected. DMRs for the detection of CRC (black curve) or the discrimination of cancer clinical stages (blue curve).

FIG. 6F illustrates a graph depicting ROC curves and their associated areas under the curve (AUC) of selected DMRs for the detection of CRC (black curve) or the discrimination of cancer clinical stages (blue curve).

FIG. 6G illustrates a graph depicting ROC curves and their associated areas under the curve (AUC) of selected DMRs for the detection of CRC (black curve) or the discrimination of cancer clinical stages (blue curve).

FIG. 6H illustrates a graph depicting ROC curves and their associated areas under the curve (AUC) of selected. DMRs for the detection of CRC (black curve) or the discrimination of cancer clinical stages (blue curve).

FIG. 6I illustrates a graph depicting ROC curves and their associated areas under the curve (AUC) of selected DMRs for the detection of CRC (black curve) or the discrimination of cancer clinical stages (blue curve).

FIG. 6J illustrates a graph depicting ROC curves and their associated areas under the curve (AUC) of selected DMRs for the detection of CRC (black curve) or the discrimination of cancer clinical stages (blue curve).

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

Table 1 is a glossary of abbreviations used in disclosure herein.

TABLE 1
Abbreviations:

| | |
|---|---|
| CEP72 | centrosomal protein 72(CEP72) |
| PDGFB | platelet derived growth factor subunit B(PDGFB) |
| GNA11 | G protein subunit alpha 11(GNA11) |
| SLC9A3 | solute carrier family 9 member A3(SLC9A3) |
| FAM20C | FAM20C, golgi associated secretory pathway |
| | kinase(FAM20C) |
| CYP2W1 | cytochrome P450 family 2 subfamily W |
| | member 1(CYP2W1) |
| SHH | sonic hedgehog(SHH) |
| WNT3 | Wnt family member 3(WNT3) |
| | histone cluster 1 H2B family member |
| HIST1H2BK | k(HIST1H2BK) |
| | SWI/SNF related, matrix associated, actin |
| | dependent regulator of chromatin, |
| SMARCD3 | subfamily d, member 3(SMARCD3) |
| | solute carrier family 2 member |
| SLC2A1 | 1(SLC2A1) |
| | hydroxyacyl-CoA |
| HADH | dehydrogenase(HADH) |
| RAPGEF2 | Rap guanine nucleotide exchange factor |
| | 2(RAPGEF2) |
| SS18L1 | SS18L1, nBAF chromatin remodeling complex |
| | subunit(SS18L1) |
| HIGD1A | HIG1 hypoxia inducible domain family member |
| | 1A(HIGD1A) |
| GNG7 | G protein subunit gamma 7(GNG7) |
| HHIPL1 | HHIP like 1(HHIPL1) |
| PHOX2B | paired like homeobox 2b(PHOX2B) |
| | protocadherin gamma subfamily A, |
| PCDHGA10 | 10(PCDHGA10) |
| SOCS3 | suppressor of cytokine signaling 3(SOCS3) |
| BAIAP2 | BAI1 associated protein 2(BAIAP2) |
| RXRA | retinoid X receptor alpha(RXRA) |
| PSD3 | pleckstrin and Sec7 domain containing |
| | 3(PSD3) |
| PTPRS | protein tyrosine phosphatase, receptor type |
| | S(PTPRS) |
| RPTOR | regulatory associated protein of MTOR complex |
| | 1(RPTOR) |
| SSTR5 | somatostatin receptor 5(SSTR5) |
| ARPC1B | actin related protein 2/3 complex subunit |
| | 1B(ARPC1B) |
| MAPK9 | mitogen-activated protein kinase 9(MAPK9) |
| KIF26A | kinesin family member 26A(KIF26A) |
| PACS2 | phosphofurin acidic cluster sorting protein |
| | 2(PACS2) |
| FGFR3 | fibroblast growth factor receptor 3(FGFR3) |
| GNAI2 | G protein subunit alpha i2(GNAI2) |
| NFKBIB | NFKB inhibitor beta(NFKBIB) |
| ELN | elastin(ELN) |
| ATP6V0CP3 | ATPase H+ transporting V0 subunit c pseudogene |
| | 3(ATP6V0CP3) succinate dehydrogenase complex |
| | assembly factor |
| SDHAF1 | 1(SDHAF1) |
| WT1 | Wilms tumor 1(WT1) |
| PLAGL1 | PLAG1 like zinc finger 1(PLAGL1) |
| TTYH3 | tweety family member 3(TTYH3) |
| ADRB3 | adrenoceptor beta 3(ADRB3) |
| KRAS | KRAS proto-oncogene, GTPase(KRAS) |
| CRB2 | crumbs 2, cell polarity complex |
| | component(CRB2) |
| GPR26 | G protein-coupled receptor 26(GPR26) |
| | ras-related C3 botulinum toxin substrate 1 |
| | (rho family, small GTP binding protein |
| RAC1 | Rac1)(RAC1) |
| | N-myc downstream regulated |
| NDRG1 | 1(NDRG1) |
| SCARB1 | scavenger receptor class B member 1(SCARB1) |
| PLXND1 | plexin D1(PLXND1) |
| ABR | active BCR-related(ABR) |
| MAP2K2 | mitogen-activated protein kinase kinase |
| | 2(MAP2K2) |
| S100A11 | S100 calcium binding protein A11(S100A11) |
| SMAD3 | SMAD family member 3(SMAD3) |
| NR4A1 | nuclear receptor subfamily 4 group A member |
| | 1(NR4A1) |
| CELSR3 | cadherin EGF LAG seven-pass G-type receptor |
| | 3(CELSR3) |
| VAV2 | vav guanine nucleotide exchange factor 2(VAV2) |
| MUC6 | mucin 6, oligomeric mucus/gel-forming(MUC6) |
| NOTCH1 | notch 1(NOTCH1) |
| EBF4 | early B-cell factor 4(EBF4) |
| RABEP1 | rabaptin, RAB GTPase binding effector protein |
| | 1(RABEP1) |
| | src-related kinase lacking C-terminal regulatory |
| SRMS | tyrosine and N-terminal myristylation sites(SRMS) |
| CACNA1B | calcium voltage-gated channel subunit alpha1 |
| | B(CACNA1B) |
| TRPV2 | transient receptor potential cation channel |
| | subfamily V member 2(TRPV2) |
| TTC28 | tetratricopeptide repeat domain 28(TTC28) |
| AKT1 | AKT serine/threonine kinase 1(AKT1) |
| DES | desmin(DES) |
| | odd-skipped related transciption |
| OSR1 | factor 1(OSR1) |
| SHC2 | SHC adaptor protein 2(SHC2) |
| | BOC cell adhesion associated, |
| BOC | oncogene regulated(BOC) |
| DPEP1 | dipeptidase 1 (renal)(DPEP1) |
| FGF3 | fibroblast growth factor 3(FGF3) |
| | Rho guanine nucleotide exchange |
| ARHGEF4 | factor 4(ARHGEF4) |
| CTBP1 | C-terminal binding protein 1(CTBP1) |
| LIMK1 | LIM domain kinase 1(LIMK1) |
| | sidekick cell adhesion molecule |
| SDK1 | 1(SDK1) |
| | Rho GTPase activating protein |
| ARHGAP23 | 23(ARHGAP23) |
| ARHGAP32 | Rho GTPase activating protein 32(ARHGAP32) |
| | dishevelled binding antagonist of beta catenin |
| DACT3 | 3(DACT3) |
| HOXC13 | homeobox C13(HOXC13) |
| GNAS | GNAS complex locus(GNAS) |
| | zinc finger and SCAN domain |
| ZSCAN18 | containing 18(ZSCAN18) |
| WNT9A | Wnt family member 9A(WNT9A) |
| | TRIO and F-actin binding |
| TRIOBP | protein(TRIOBP) |
| ESPNL | espin-like(ESPNL) |
| | necdin, MAGE family |
| NDN | member(NDN) |
| CYTH2 | cytohesin 2(CYTH2) |
| CDH4 | cadherin 4(CDH4) |
| | collagen type IX alpha 3 |
| COL9A3 | chain(COL9A3) |
| | collagen type XXVII alpha 1 |
| COL27A1 | chain(COL27A1) |
| PLCD4 | phospholipase C delta 4(PLCD4) |
| RASA3 | RAS p21 protein activator 3(RASA3) |
| COL4A2 | collagen type IV alpha 2 chain(COL4A2) |
| | alpha 1,4- |
| A4GALT | galactosyltransferase(A4GALT) |
| | ATPase Na+/K+ transporting subunit |
| ATP1A3 | alpha 3(ATP1A3) |
| RALGDS | ral guanine nucleotide dissociation |
| | stimulator(RALGDS) |
| LSP1 | lymphocyte-specific protein 1(LSP1) |
| LEP | leptin(LEP) |
| | solute carrier family 17 member |
| SLC17A7 | 7(SLC17A7) |
| | LDL receptor related protein |
| LRP1 | 1(LRP1) |
| FBLN2 | fibulin 2(FBLN2) |
| NFIC | nuclear factor I C(NFIC) |
| | ArfGAP with RhoGAP domain, |
| | ankyrin repeat and PH domain |
| ARAP1 | 1(ARAP1) |

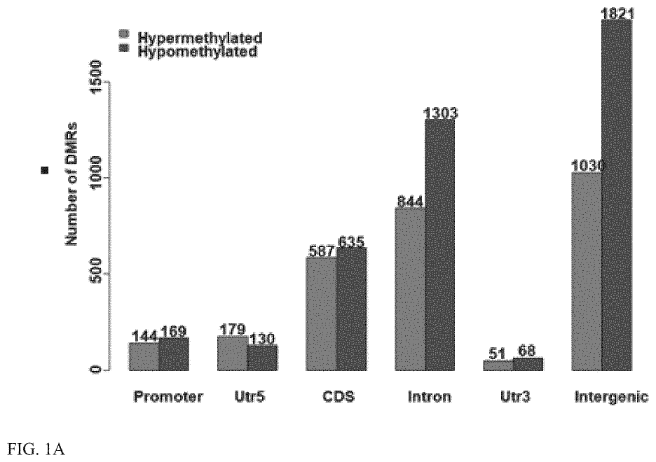

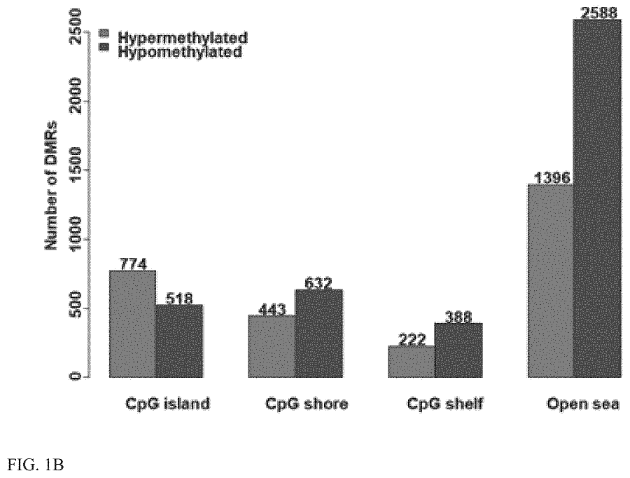

According to an aspect, the innovation provides a method to identify biomarkers for blood-based early detection of CRC using genome-wide methylation sequencing data to detect DNA methylation alterations in peripheral blood of CRC patients or suspected CRC patients (see FIGS. 1A and 1B). Altered distribution patterns of DMRs, such as global hypomethylation and hypermethylation of CpG islands can be observed, suggesting abnormal DNA methylation patterns can be detected in peripheral blood. DNA methylation alterations in peripheral blood may represent an early response to the presence of tumor or reflect only tumor-derived changes.

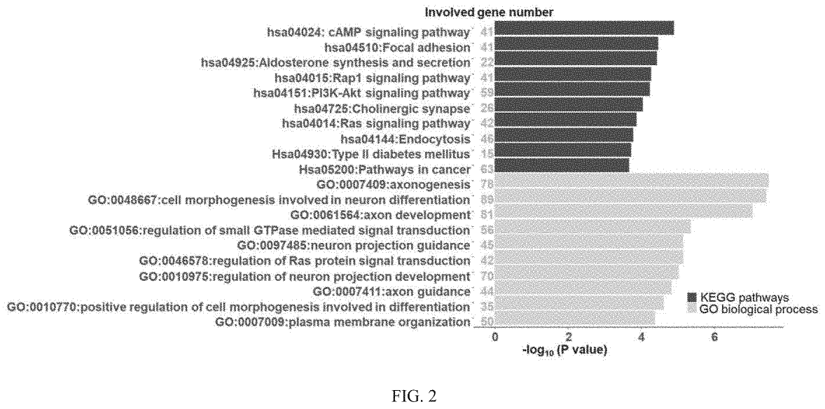

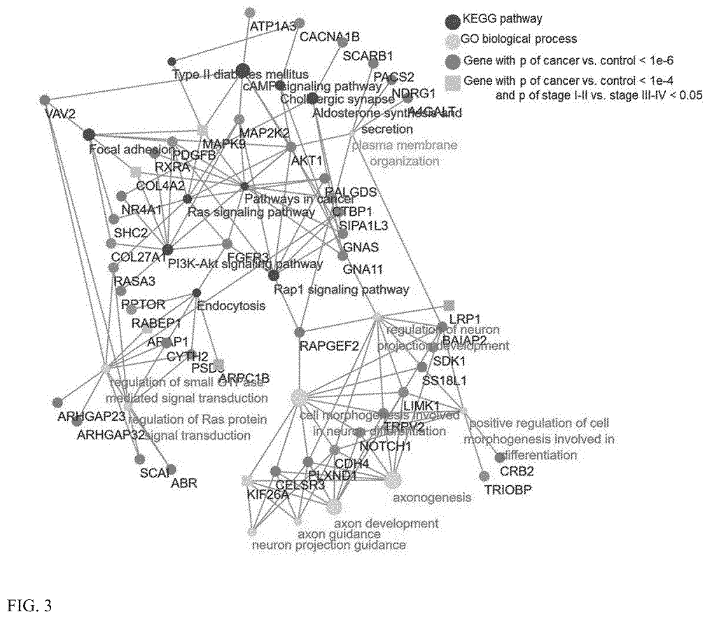

Functional analyses revealed that DNA methylation alterations in peripheral blood contribute to the activation of Ras signaling which is involved in CRC initiation and development (FIGS. 2 and 3). In the Ras signaling pathway, most DMRs located in the genebody region were hypermethylated. Activation of FGFR proteins can lead to the activation of the RAS-MAPK pathway and the PI3K-AKT pathway. RAPGEF2 serve as RAS activators by promoting the acquisition of GTP to maintain the active GTP-bound state and is the key link between cell surface receptors and RAS activation. Without being bound by theory, because methylation in gene bodies is generally associated with increased gene expression, it is believed that DNA methylation alterations contribute to the activation of Ras signaling and colorectal tumorigenesis.

Most DMRs associated with genebody regions of Rac-related genes can be hypomethylated. DNA methylation alterations can inhibit Rac signaling, which is an important regulator of Arp2/3-dependent actin polymerization and phagocytosis of invading pathogens. DNA methylation alterations can be in the endocytosis pathway (FIGS. 2 and 3). In one embodiment, an innate immune response mechanism is the ingestion of extracellular macromolecules through endocytosis or phagocytosis of bacteria in order to remove the inflammatory stimuli. DNA methylation alterations in peripheral blood of patients with CRC may compromise the ability of intestinal epithelial cells to respond to invading pathogens. Changes in the amount and composition of collagen can contribute to the disruption of intestinal mucus and promote bacterial invasion, inflammation, and lead to the development of cancer.

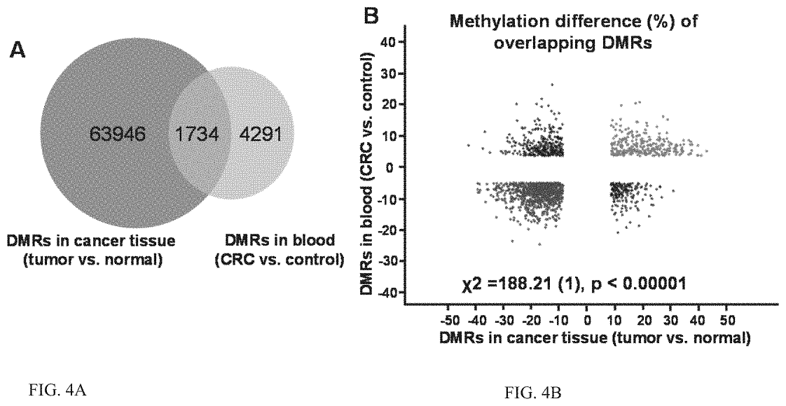

The method can reveal key functional genes with significant aberrant DNA methylation during carcinogenesis using genome-wide methylation analysis on CRC tumors (N=10) and comparing to adjacent normal tissues (N=10). In a test embodiment, ages and genders of tissue donors were comparable to blood donors. 65,680 DMRs were identified between CRC tumor and normal tissues and compared to 6,025 DMRs between CRC peripheral blood and healthy controls. To evaluate whether DNA methylation alteration in peripheral blood is associated with CRC tumor, we overlapped these DMRs identified separately from CRC tumor and peripheral blood (FIGS. 4A and 4B). If tumor and peripheral blood DNAs were both differentially methylated at a certain CpG site, the DMC was counted as an overlapping DMR. There were 1,734 overlapping DMRs, accounting for 28.8% of DMRs identified from the peripheral blood group (FIG. 4A). We observed methylation changes of these overlapping DMRs occurred in the same direction for peripheral blood and tumor. In other words, a majority of overlapping DMRs for peripheral blood and tumor were either hypermethylated (17.3%) or hypomethylated (52.8%). A Chi-square test showed that there was a significant association between DMRs in peripheral blood and tumor (FIG. 4B, p<0.00001).

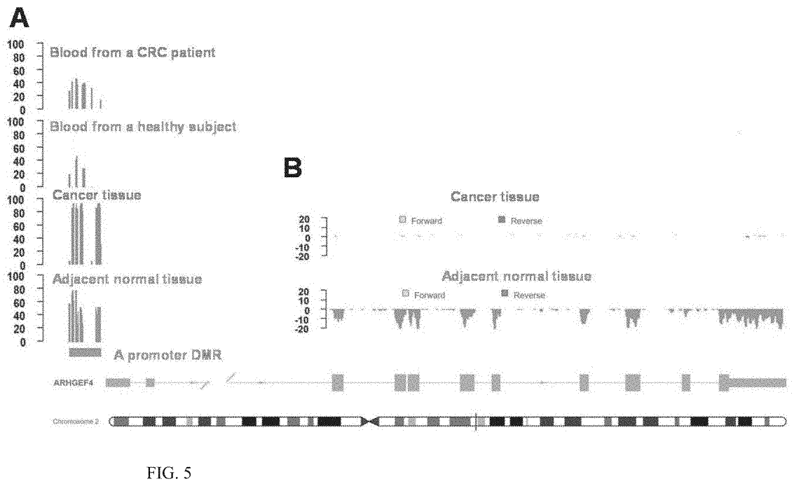

The method employs RNA-Seq data (N=10) from the same CRC tissue donors to determine transcriptome-wide changes in CRC, compared to adjacent normal tissues. In a test embodiment, the method was used to perform an integrated analysis between the transcriptome and methylation profile in CRC to reveal the DNA methylation changes and the underlying regulatory mechanisms of the impact of DNA methylation alteration on gene expression during CRC development. The method determines genes having overlapping DMRs in blood and tumors and selects altered gene expression as candidate biomarkers. For example, APC-stimulated guanine nucleotide-exchange factor (ARHGEF4) is a binding partner of adenomatous polyposis coli (APC), which is an important tumor suppressor gene of great importance in the development of CRC. The method determines that ARHGEF4 gene was hypermethylated in the promoter region of both in CRC peripheral blood and tumors (FIG. 5A), and the gene expression was silenced in tumor samples (FIG. 5B). For example, the results suggest that ARHGEF4 promoter hypermethylation may be involved in tumor suppressor APC inactivation.

The method evaluates the accuracy of genes with DMRs for early detection of CRC. In one example embodiment, the method calculated receiver operating characteristic (ROC) curves and areas under the ROC curve (AUCs) of the selected DMRs. According to an aspect, genes associated with overlapping DMRs in blood and tumors and have altered gene expression can be selected by the method as potential candidate biomarkers as shown in Table 3.

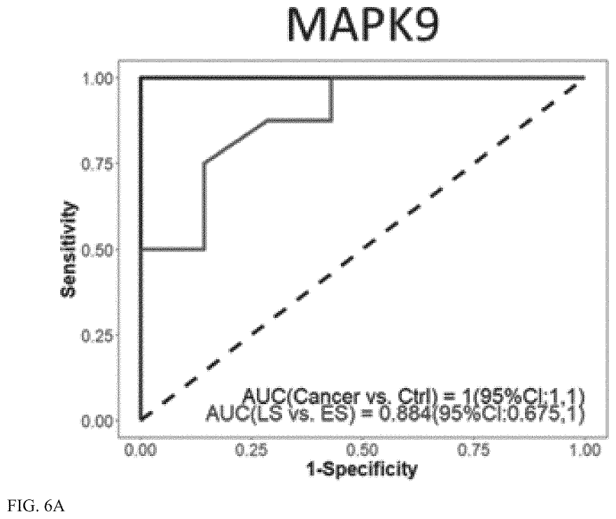

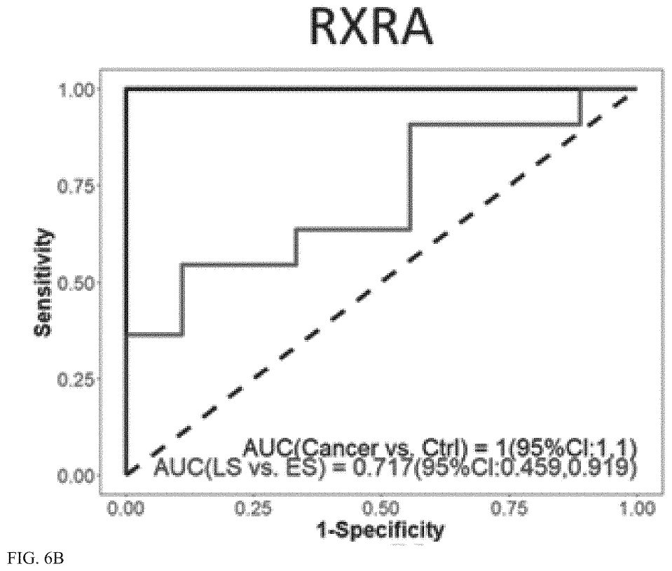

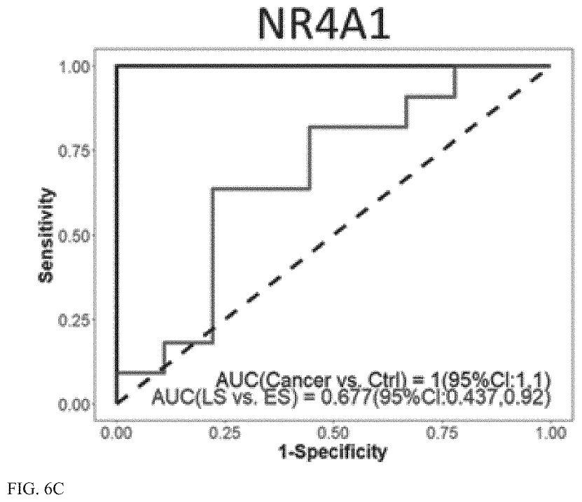

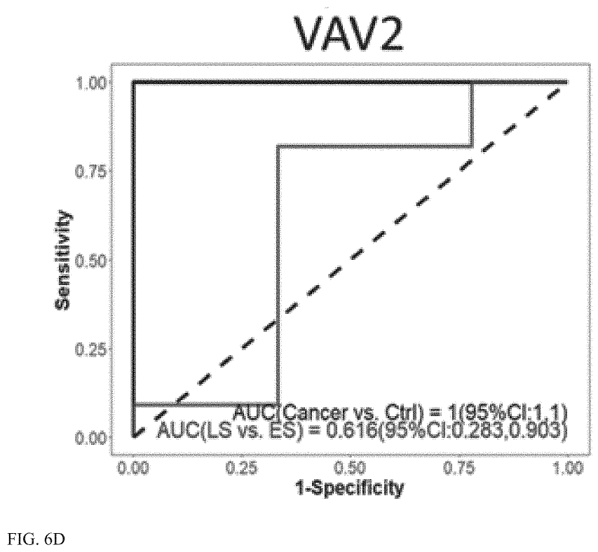

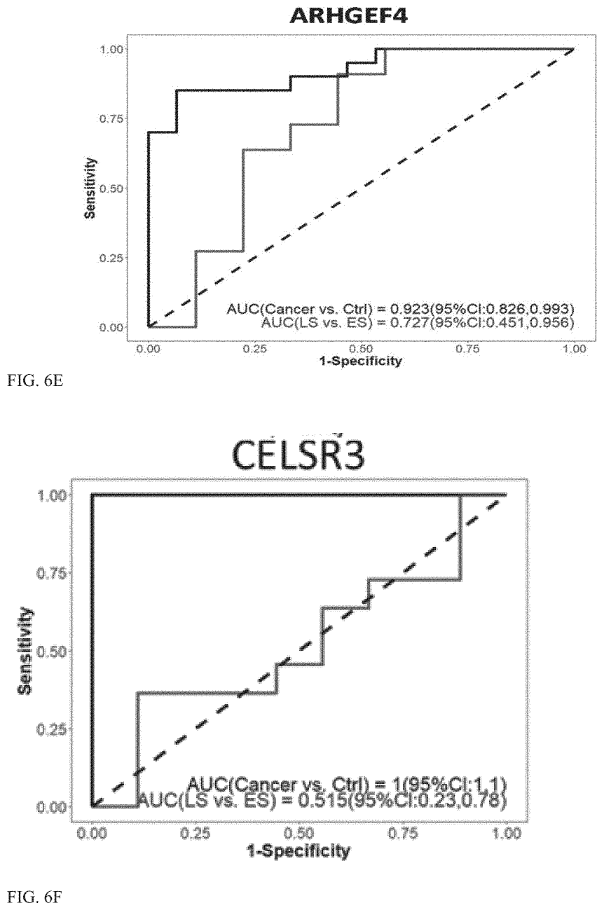

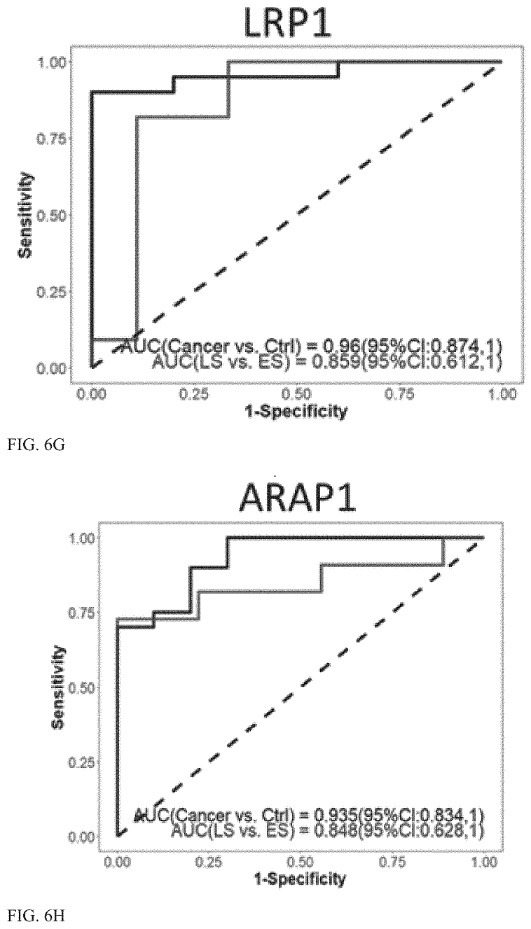

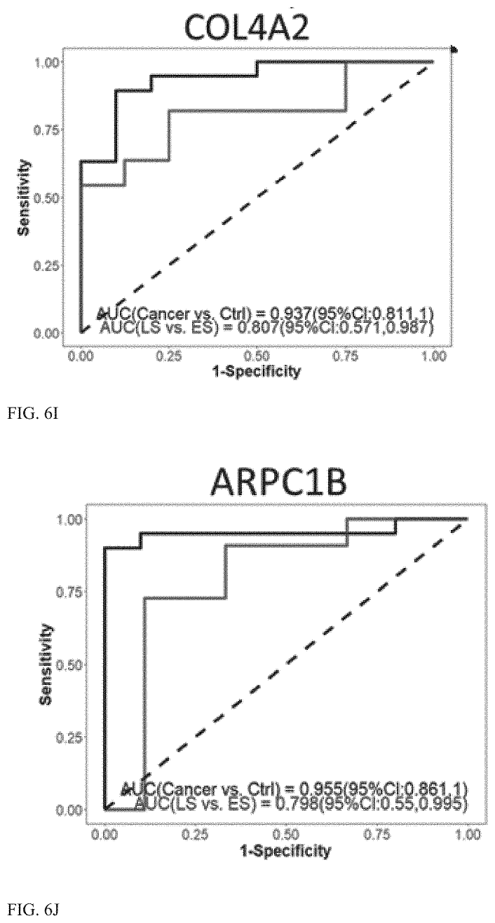

To evaluate the accuracy of genes with DMRs for early detection of CRC, the method calculates receiver operating characteristic (ROC) curves and areas under the ROC curve (AUCs) of the selected DMRs. For example, genes including MAPK9, RXRA, NR4A1, VAV2, ARHGEF4 and CELSR3 exhibited significant accuracy for the detection of CRC [AUC of CRC vs. control=1(>92% CI: 1, 1) for all DMRs] (FIG. 6A-6F). In one embodiment, genes including MAPK9, LRP1, ARAP1, COL4A2 and ARPC1B can discriminate CRC from early-stage to late-stage cancer in the peripheral blood DNA [AUC of late stage vs. early stage=0.884 (95% CI: 0.675, 1), 0.859 (95% CI: 0.612, 1), 0.848 (95% CI: 0.628, 1), 0.807 (95% CI: 0.571, 0.987) and 0.798 (95% CI: 0.55, 0.995), respectively] (FIG. 6G-6J). A completed list of gene panels are detailed in Table 3.

### Example

In an exemplary embodiment, the method analyzes mechanisms and cell regulatory effects of DNA methylation alterations in CRC were investigated.

Study Population.

Whole blood samples (n=20) from CRC patients were obtained from the Cooperative Human Tissue Network (CHTN) (Table 2). The DNA methylation data of whole blood samples from healthy controls (n=10) was obtained from a publicly available database (NCBI GEO; accession number GSE85928). Paired CRC tumor and adjacent normal tissue from 10 age and gender-matched CRC patients were also used.

TABLE 2
General characteristics of healthy subjects and CRC patients

| Characteristics | Healthy subjects (N = 10) | CRC patients (N = 20) |
|---|---|---|
| Age (years) | | |
| Mean ± SD | 30 ± 7 | 63 ± 11 |
| Range | 21-43 | 38-86 |
| Gender | | |
| Male | 5 | 7 |
| Female | 5 | 13 |
| Clinical stage | | |
| I-II | — | 9 |
| III-IV | — | 11 |

DNA Extraction, RRBS Library Preparation and Sequencing.

An RRBS library was prepared. Genomic DNA was digested overnight with Msp1 (New England Biolabs, USA) followed by end-repair and ligation of sequencing adaptors. A DNA library was prepared using NEXTflex Bisulfite-Seq Kit (Bioo Scientific) following a standard procedure. Bisulfite conversion of non-methylated cytosines was performed using the EZ DNA Methylation-Gold kit (Zymo Research Corp.) following the manufacturer's instructions. All PCR reactions for RRBS were purified using AMPure XP (Beckman Coulter, Brea, USA), and analyzed on a bioanalyzer. Sequencing was performed on the Illumina HiSeq2500 for a paired-end 2×50 bp run, with 150 million reads from each direction. Data quality checking was done on the Illumina SAV. De-multiplexing was performed with the Illumina Bcl2fastq2 v2.17 program.

Bioinformatics and Statistical Analysis.

The quality of the raw reads was examined with FastQC. The adapter trimming and filtering of the high quality reads was carried out with Cutadapt v1.8.3 and Trim Galore v0.4.0 with the—RRBS option. Quality processed reads were mapped to human genome (hg19) using Bismark assisted by Bowtie2. Before DMC and DMR analyses, methylation calls were filtered by discarding bases with coverage below 5× and bases with more than 99.9^{th} percentile coverage in each sample. CpG sites on sex chromosomes and mitochondrion were excluded from the analyses. Individual DMCs were identified between CRC and control groups using logistic regression with the R package methylKit. Read coverage was normalized between samples. A minimum of three individuals per group were required for a CpG site to be analyzed. The CpGs with at least 10% methylation difference and a q-value <0.05 were considered to be differentially methylated. DMRs were determined using the R package eDMR with default parameters. To be considered significant, a DMR needed to contain at least one DMC, three CpG sites, and an absolute mean methylation difference greater than 5%. The DMRs identified using UCSC Refseq gene models with promoter regions defined as being 2 kb upstream from the transcription start site (TSS) were annotated. CpG islands were defined based on UCSC annotation (http://genome.ucsc.edu/). Functional Gene Ontology (GO) and Kyoto Encyclopedia of Genes and Genomes (KEGG) pathway enrichment analyses of involved genes were performed using DAVID bioinformatics resources (version 6.8; http://david.abcc.ncifcrf.gov/). The p-value was calculated using the modified Fishers exact test and the GO categories and KEGG pathways were identified as significantly enriched when p value was <0.05. Additional parameters were set to the default values. The Mann-Whitney U test was used for comparisons between the groups of subjects. A p value <0.05 was defined as statistically significant. The ROC curves were constructed and the areas under the ROC curves (AUCs) were calculated to evaluate the accuracy of DMRs for predicting CRC. The bootstrap method with 500 bootstrap samples was used to obtain the 95% confidence interval (CI) of the AUC. A 95% CI of AUCs not including 0.5 indicates a significant result.

Distribution of DMRs Identified in CRC.

Through differential methylation analysis, 6961 DMRs between CRC vs. control were identified. These DMRs to gene regions (FIG. 1A) and CpG islands (FIG. 1B) were annotated. To avoid systematic errors for the DNA methylation data, histogram transformation was applied to equalize the distributions of the methylation levels to the control group. Age and gender were used as covariates in differential analysis in order to remove those possible effects. At the gene context based on genomic content, peripheral blood showed higher overall genomic hypomethylation than hypermethylation. This is consistent with previous reports showing that genomic DNA hypomethylation is a hallmark of most cancer genomes, prompting genomic instability and cancer transformation. The DMR distribution showed that more hypermethylated DMRs were located in CpG islands.

Functional Analysis of Genes Associated with DMRs.

These DMRs were further selected using non-parametric methods. The average methylation level across DMRs was calculated for each subject. The Mann-Whitney U test was then performed to determine significant differences between CRC and control groups, and differences between early (stage I and II) and late stage (stage III and IV) CRC patients. 1,852 DMRs located in the promoter or gene body regions with significant differences in mean methylation levels between the CRC and control groups were located.

Kyoto Encyclopedia of Genes and Genomes (KEGG) pathway and Gene Ontology (GO) biological process (GO-BP) analyses of the 1,852 genes associated with selected DMRs were used to understand how DNA methylation contributes to CRC development. The top 10 KEGG pathways and top 10 GO-BPs are summarized in FIG. 2. The top 10 significantly enriched KEGG pathways include: the cAMP signaling pathway, focal adhesion, aldosterone synthesis and secretion, Rap1 signaling pathway, PI3K-Akt signaling pathway, cholinergic synapse, Ras signaling pathway, endocytosis, type II diabetes mellitus and pathways in cancer.

The top 10 significantly enriched GO biological processes include: axonogenesis, cell morphogenesis as involved in neuron differentiation, axon development, regulation of small GTPase-mediated signal transduction, neuron projection guidance, regulation of Ras protein signal transduction, regulation of neuron projection development, axon guidance, positive regulation of cell morphogenesis involved in differentiation and plasma membrane organization.

DNA Methylation Alterations of RAS Signaling Pathways.

It was observed that most of the DMRs that were associated with the genebody regions of Ras-related genes were hypermethylated. For example, in the Ras signaling pathway (hsa04014), fibroblast growth factor receptor 3 (FGFR3) and rap guanine nucleotide exchange factor 2 (RAPGEF2), upstream of RAS, were associated with hypermethylated DMRs in their coding sequence (CDS) and intron region, respectively. Ral guanine nucleotide dissociation stimulator (RALGDS) and mitogen-activated protein kinase kinase 2 (MAP2K2), downstream of RAS, were associated with hypermethylated DMRs in their intron regions. Ras GTPase-activating protein 3 (RASA3), a negative regulator of the Ras signaling pathway, was associated with a hypomethylated DMR in the intron region.

Most DMRs which are associated with the genebody regions of Rac-related genes were hypomethylated. Rac family small GTPase 1 (Rac1) is one of the key signaling components to control actin cytoskeleton organization and to suppress endometrial cancer metastasis. The Rac1-actin-related protein 2/3 (Arp2/3) pathway plays a critical role in phagocytosis of invading pathogens through cytoskeletal rearrangements. Guanine nucleotide exchange factor (VAV2), upstream of the Rac1, was associated with a hypomethylated DMR in the CDS region. Although not on the top of the DMR list, Rac1 is associated with a hypomethylated DMR (p of CRC vs. control=4.38×10⁻⁵) in the CDS region. The downstream genes, LIM-kinase1 (LIMK1), BAI1 associated protein 2 (BAIAP2) and Arp2/3 Complex Subunit 1B (ARPC1B) were associated with a hypomethylated DMR in their CDS, intron and CDS region, respectively.

It was also observed that DNA methylation was altered in the endocytosis pathway (hsa04144). Rabaptin, Rab GTPase binding effector protein 1 (RABEP1), was associated with a hypermethylated DMR in the intron region. Cytohesin 2 (CYTH2) was associated with a hypermethylated DMR in the CDS region. Pleckstrin and sec7 domain containing 3 (PSD3) was associated with a hypomethylated DMR in the intron region. ArfGAP with RhoGAP domain, ankyrin repeat and PH domain 1 (ARAP1) was associated with a hypomethylated DMR in the CDS region.

DNA Methylation Alterations in Other Cancer-related Pathways.

In the PI3K-Akt signaling pathway (hsa04151), AKT serine/threonine kinase 1 (AKT1) was associated with a hypomethylated DMR in the CDS region. Regulatory associated protein of mTOR complex 1 (RPTOR) was associated with a hypermethylated DMR in the intron region. Retinoid X receptor alpha (RXRA) and its heterodimerization partner, nuclear receptor subfamily 4 group A member 1 (NR4A1), exhibits pro-oncogenic activity and enhances either survival and/or cell proliferation. NR4A1 was associated with a hypomethylated DMR in the 5′ UTR region and RXRA was associated with a hypermethlated DMR in the intron region.

Notch signaling is overexpressed or constitutively activated in many cancers including CRC. Overexpression of C-terminal binding protein 1 (CTBP1) contributes to colon adenoma initiation and has been reported to be associated with poor prognosis, consistently, in the Notch signaling pathway (hsa04330), it was observed that Notch homolog 1 (NOTCH1) and CTBP1 were associated with hypermethylated DMRs in their CDS regions. In the MAPK signaling pathway (hsa04010), Mitogen-activated protein kinase 9 (MAPK9) is a member of the MAP kinase family and blocks the ubiquitination of tumor suppressor p53. A hypomethylated DMR in the intron region was observed. Abnormal choline metabolism may be a metabolic hallmark associated with oncogenesis and tumour progression. There were 6 genes involved in the choline metabolism in cancer (hsa05231) including AKT1, MAPK9, MAP2K2, RALGDS, RAC1 and platelet derived growth factor (PDGFB). There were 3 genes involved in the calcium signaling pathway (hsa04020) including calcium voltage-gated channel subunit α1B (CACNA1B), guanine nucleotide-binding protein G subunit alpha (GNAS) and guanine nucleotide-binding protein subunit alpha-11 (GNA11). Moreover, RXRA (described above) is also an important genetic pathway in the calcium/vitamin D pathway.

In addition, some selected DMRs are associated with genes that are related to CRC development. Plexin D1 (PLXND1) mediates anti-angiogenic signaling and recent findings suggest it is upregulated in CRC. A hypermethylated DMR in its CDS region was observed. Upregulation of N-Myc downstream regulated 1 (NDRG1) has been associated with poor prognosis in CRC, and the data suggests that it is associated with a hypermethylated DMR in the intron region. LDL receptor-related protein 1 (LRP1) mediates the clearance of many extracellular enzymes involved in the spread of cancer cells: metalloproteinases and serine proteinases. Decrease of LRP1 activity or loss of LRP1 expression correlates with increased aggressiveness of cancer cells in certain types of cancer. A hypomethylated DMR in the CDS region was observed. Collagen type IV a 2 chain (COL4A2) encodes one of the six subunits of type IV collagen, the major structural component of basement membranes. It also functions as an inhibitor of angiogenesis and tumor growth. Consistent with previous findings, a hypomethylated DMR in the CDS region of COL4A2 was observed.

In order to reveal specific functional genes with significant aberrant DNA methylation during carcinogenesis, genome-wide methylation analysis was conducted in CRC tumors (N=10) compared to adjacent normal tissues (N=10). The age and gender of tissue donors were comparable to blood donors. 65,680 DMRs were identified between CRC tumor and normal tissues and compared to 6,025 DMRs between CRC peripheral blood and healthy controls. To evaluate whether DNA methylation alteration in peripheral blood is associated with CRC tumor, these DMRs identified separately from CRC tumor and peripheral blood were overlapped (FIG. 4). If tumor and peripheral blood DNAs were both differentially methylated at a certain CpG site, the DMC was counted as an overlapping DMR. There were 1,734 overlapping DMRs, accounting for 28.8% of DMRs identified from the peripheral blood group (FIG. 4A). Methylation changes of these overlapping DMRs occurred in the same direction for peripheral blood and tumor. A majority of overlapping DMRs for peripheral blood and tumor were either hypermethylated (17.3%) or hypomethylated (52.8%). A Chi-square test showed that there was a significant association between DMRs in peripheral blood and tumor (FIG. 4B, p<0.00001).

RNA-Seq data (N=10) from the same CRC tissue donors was then used to determine the transcriptome-wide changes in CRC, compared to adjacent normal tissues. Integrated analysis between the transcriptome and methylation profile in CRC were performed to reveal the DNA methylation changes and the underlying regulatory mechanisms of the impact of DNA methylation alteration on gene expression during CRC development. Genes with overlapping DMRs in blood and tumors and have altered gene expression were selected as potential candidate biomarkers. This integrated analysis methods allowed us to efficiently map tumor-specific DNA methylation alterations in whole blood and tumor with an accompanying gene expression change. The average methylation levels in tumor tissue and in blood across overlapping DMRs was calculated for each subject. Spearman correlation coefficient can be used to determine the correlation of methylation levels of overlapping DMRs between tumor and blood DNA and hierarchical clustering can be used to identify significantly correlated DMRs. Significantly correlated DMRs are therefore a potential measure of the degree of association between the DNA methylation alterations in blood and tumor tissue.

Using integrated RRBS and RNA-Seq, 96 genes associated with overlapping DMRs were screened as listed in Table 3. For example, APC-stimulated guanine nucleotide-exchange factor (ARHGEF4) is a binding partner of adenomatous polyposis coli (APC), which is a tumor suppressor gene of importance in the development of CRC. The method found that the ARHGEF4 gene was hypermethylated in t-he promoter region of both in CRC peripheral blood and tumors (FIG. 2A), and the gene expression was silenced in tumor samples (FIG. 2B) suggesting that ARHGEF4 promoter hypermethylation may be involved in tumor suppressor APC inactivation.

Accuracy of DMRs for Detecting CRC and Discriminating Early-stage Patients.

To evaluate the accuracy of DMRs for early detection of CRC, receiver operating characteristic (ROC) curves and areas under the ROC curve (AUCs) of the selected DMRs were calculated. Table 3 shows the ROC results showing the ability of these DMRs to detect CRC and to differentiate early-stage cancer. Overall 96 genes associated with overlapping DMRs were are identified by the method. Either DMRs with mean methylation levels were increased or decreased progressively over control, or can be used to differentiate CRC early stage from late stage. For example, DMRs associated with MAPK9, RXRA, NR4A1, VAV2 and CELSR3 have a high ability to detect CRC [AUC of CRC vs. control=1(95% CI: 1, 1) for all DMRs]. DMRs associated with MAPK9, LRP1, ARAP1, COL4A2 and ARPC1B have a high ability to differentiate early-stage cancer [AUC of late stage vs. early stage=0.884 (95% CI: 0.675, 1), 0.859 (95% CI: 0.612, 1), 0.848 (95% CI: 0.628, 1), 0.807 (95% CI: 0.571, 0.987) and 0.798 (95% CI: 0.55, 0.995), respectively].

TABLE 3
Summary of selected DMRs.

| | | | | | | |
|---|---|---|---|---|---|---|
| | | | | LS Mean | ES Mean | CTRL Mean |
| DMR Location | Gene | Gene Regions | CpG Island | (SD) | (SD) | (SD) |

Hypermethylated

| | | | | | | |
|---|---|---|---|---|---|---|
| chr3: 129276017-129277607 | PLXND1 | CDS | open sea | 72.98 (3.09) | 75.35 (3.21) | 61.04 (3.75) |
| chr4: 1800772-1801634 | FGFR3 | CDS | CpG island | 76.34 (3.26) | 78.09 (3.64) | 60.66 (4.22) |
| chr4: 1219063-1219488 | CTBP1 | CDS | CpG island | 84.09 (1.94) | 83.9 (1.65) | 74.96 (5.29) |
| chr4: 160256911-160257058 | RAPGEF2 | intron | open sea | 62.71 (3.44) | 63.16 (3.72) | 42.51 (12.46) |
| chr8: 134279628-134280406 | NDRG1 | intron | open sea | 65.17 (3.23) | 66.72 (3.5) | 55.33 (3.93) |
| chr9: 117063843-117064728 | COL27A1 | CDS | open sea | 51.47 (2.73) | 54.03 (3.03) | 39.52 (4.89) |
| chr9: 139411694-139412139 | NOTCH1 | CDS | Shore | 72.11 (3.14) | 70.68 (3.77) | 63.65 (1.24) |
| chr9: 136003298-136003875 | RALGDS | intron | open sea | 59.4 (4.91) | 61.11 (4.21) | 35.13 (6.31) |
| chr9: 137324620-137325166 | RXRA | intron | open sea | 59.97 (4.33) | 56.81 (3.95) | 39.89 (6.44) |
| chr9: 127831756-127831995 | SCAI | intron | open sea | 75.18 (4.16) | 76.32 (5.4) | 63.17 (4.23) |
| chr9: 126123252-126123504 | CRB2 | intron | open sea | 59.09 (5.84) | 60.48 (6.55) | 35.6 (11.5) |
| chr11: 128839732-128840325 | ARHGAP32 | CDS | open sea | 72.46 (2.48) | 72.38 (3.4) | 58.54 (8.92) |
| chr17: 16329581-16329742 | TRPV2 | CDS | open sea | 71.7 (2.87) | 71.98 (2.47) | 55.44 (12.96) |
| chr17: 78836096-78836514 | RPTOR | intron | open sea | 68.05 (2.25) | 66.79 (3.12) | 57.33 (4.35) |
| chr17: 5207615-5207733 | RABEP1 | intron | open sea | 72.75 (2.68) | 72.29 (5.02) | 65.03 (2.72) |
| chr19: 48981419-48981798 | CYTH2 | CDS | Shore | 78.88 (1.48) | 79.57 (2.63) | 52.43 (24.14) |
| chr19: 38673435-38674132 | SIPA1L3 | intron | open sea | 78.09 (2.36) | 79.1 (3.85) | 62.7 (10.15) |
| chr19: 4101341-4101586 | MAP2K2 | intron | CpG island | 78.5 (2.91) | 77.4 (2.82) | 67.08 (14.01) |
| chr20: 57464965-57465494 | GNAS | promoter | CpG island | 41.19 (3.4) | 39.92 (2.05) | 23.96 (11.88) |
| chr20: 60737881-60738663 | SS18L1 | CDS | CpG island | 76.67 (2.82) | 78.91 (1.83) | 61.12 (10.65) |
| Chr19: 58629653-58629967 | ZSCAN18 | 5′ UTR | CpG island | 18.14 (4.47) | 18 (3.33) | 8.95 (4.87) |
| Chr22: 29075690-29076384 | TTC28 | 5′ UTR | CpG island | 22.96 (8.61) | 21.26 (5.43) | 6.71 (3.46) |
| Chr7: 127881049-127881369 | LEP | 5′ UTR | CpG island | 25.61 (4.4) | 22.87 (5.39) | 14.32 (6.51) |
| Chr2: 131673573-131674131 | ARHGEF4 | Promoter | CpG island | 26.28 (4.38) | 22.06 (6.3) | 14.83 (5.13) |
| Chr5: 140792683-140793474 | PCDHGA10 | promoter | open sea 2 | 17.75 (3.74) | 14.01 (4.49) | 7.93 (4.59) |
| Chr6: 42695094-42695626 | ATP6V0CP3 | 5′ UTR | CpG island | 94.91 (2.13) | 93.46 (4.18) | 20.65 (24.97) |
| Chr19: 39398053-39398350 | NEKBIB | 5′ UTR | CpG island | 84.04 (4.67) | 85.41 (9.13) | 72.19 (11.66) |
| Chr6: 144329245-144329549 | PLAGL1 | 5′ UTR | CpG island | 35.57 (2.19) | 34.72 (1.91) | 26.98 (8.08) |
| Chr12: 54331986-54333458 | HOXC13 | 5′ UTR | CpG island | 13.96 (4.04) | 11.86 (2.06) | 8.34 (3.49) |
| Chr19: 47152581-47152993 | DACT3 | CDS | CpG island | 12.56 (3.71) | 12.71 (2.3) | 6.23 (3.16) |
| Chr7: 1028328-1028614 | CYP2W1 | CDS | Shore | 20.25 (6.86) | 18.65 (4.34) | 8.7 (4.59) |
| Chr7: 155595151-155596289 | SHH | CDS | CpG island | 11.62 (1.95) | 12.04 (1.68) | 8.02 (2.87) |
| Chr8: 37823708-37823868 | ADRB3 | CDS | CpG island | 20.07 (6.62) | 16.25 (3.9) | 9.68 (5.53) |
| Chr19: 49936977-49937165 | SLC17A7 | CDS | CpG island | 18.41 (2.45) | 17.76 (3.2) | 10.73 (4.75) |
| Chr15: 23931943-23932081 | NDN | CDS | CpG island | 36.73 (5.98) | 33.74 (2.92) | 24.5 (9.43) |
| Chr7: 2691893-2692388 | TTYH3 | Intron | Open sea | 56.96 (3.91) | 55.7 (3.32) | 47.05 (15.93) |
| Chr11: 32452200-32453076 | WT1 | 5′ UTR | CpG island | 24.29 (8.81) | 22.85 (2.36) | 15.38 (8.89) |
| Chr2: 19553173-19553431 | OSR1 | CDS | CpG island | 27.05 (4.67) | 27.82 (2.68) | 20.51 (6.61) |
| Chr20: 2730191-2730750 | EBF4 | CDS | CpG island | 45.22 (5.59) | 46.18 (3.56) | 28.38 (15.97) |
| Chr10: 125425607-125426325 | GPR26 | 5′ UTR | CpG island | 18.08 (3.38) | 16.28 (3.47) | 11.7 (4.31) |
| Chr4: 41747769-41748363 | PHOX2B | CDS | CpG island | 16.87 (4.78) | 13.22 (5.86) | 8.64 (4.37) |
| Chr14: 100126403-100126679 | HHIPL1 | CDS | CpG island | 13.21 (8.02) | 11.26 (3.05) | 7.1 (3.22) |
| Chr5: 624972-625147 | CEP72 | Intron | Shelf | 66.22 (3.77) | 66.07 (4.65) | 47.12 (23.91) |
| Chr16: 89690046-89690262 | DPEP1 | Intron | Open sea | 40.11 (14.86) | 42.56 (7.38) | 28.71 (10.25) |
| Chr19: 3452488-3452664 | NFIC | CDS | CpG island | 78.59 (2.62) | 74.16 (4.51) | 67.2 (18.27) |
| Chr2: 220283249-220283951 | DES | CDS | CpG island | 13.98 (1.88) | 13.5 (2.87) | 7.16 (3.85) |

Hypomethylated

| | | | | | | |
|---|---|---|---|---|---|---|
| chr3: 48677529-48678331 | CELSR3 | CDS | CpG island | 38.93 (3.53) | 39.36 (3.19) | 54.24 (3.19) |
| chr5: 179695549-179695862 | MAPK9 | intron | open sea | 34.04 (14.83) | 46.56 (3.91) | 57.05 (2.07) |
| chr7: 98984785-98986056 | ARPC1B | CDS | open sea | 57.5 (2.78) | 61.12 (3.46) | 66.1 (2.26) |
| chr7: 73520260-73520947 | LIMK1 | CDS | open sea | 59.11 (3.08) | 57.71 (2.76) | 68.92 (3.95) |
| chr7: 4208636-4208932 | SDK1 | intron | open sea | 53.74 (3.18) | 55.19 (2.17) | 64.82 (4.99) |
| chr8: 18827759-18827909 | PSD3 | intron | open sea | 63.43 (3.99) | 63.71 (5.53) | 75.7 (2.43) |
| chr9: 136669856-136671417 | VAV2 | CDS | open sea | 55.46 (1.83) | 56.55 (2.6) | 63.6 (2.75) |
| chr9: 141013037-141013769 | CACNA1B | CDS | Shore | 35.04 (3.78) | 37.17 (6.93) | 55.53 (4.82) |
| chr11: 72404012-72404706 | ARAP1 | CDS | Shelf | 53.45 (2.08) | 55.93 (1.15) | 58.28 (1.85) |
| chr12: 52447977-52448157 | NR4A1 | 5′ UTR | Shelf | 43.34 (3.95) | 45.39 (3.78) | 60 (6.37) |
| chr12: 57590785-57591112 | LRP1 | CDS | open sea | 20.87 (9.9) | 41.66 (14.44) | 57.53 (6.14) |
| chr12: 125296703-125297291 | SCARB1 | intron | Shelf | 61.66 (2.43) | 61.86 (3.18) | 71.85 (3.29) |
| chr13: 111117540-111117853 | COL4A2 | CDS | open sea | 43.53 (7.86) | 53.18 (5.55) | 60.56 (3.9) |
| chr13: 114823604-114823726 | RASA3 | intron | open sea | 44.63 (5.95) | 42.37 (7.81) | 58.75 (2.35) |
| chr14: 105239801-105240179 | AKT1 | CDS | CpG island | 55.58 (3.29) | 53.7 (2.95) | 67.92 (3.48) |
| chr14: 104612850-104614023 | KIF26A | intron | open sea | 57.51 (4.17) | 60.65 (1.66) | 65.23 (3.04) |
| chr14: 105831329-105832002 | PACS2 | intron | Shore | 41.5 (3.41) | 40.17 (3.73) | 50.92 (1.17) |
| chr17: 958859-960394 | ABR | CDS | open sea | 28.46 (7.71) | 28.76 (7.1) | 46.14 (4.33) |
| chr17: 36638843-36639223 | ARHGAP23 | CDS | open sea | 59.68 (5.15) | 61.77 (6.59) | 81.41 (5.62) |
| chr17: 79074960-79075309 | BAIAP2 | intron | Shelf | 37.89 (2.89) | 36.72 (3.08) | 49.19 (4.18) |
| chr19: 42492254-42492761 | ATP1A3 | CDS | open sea | 70.51 (6.76) | 70.86 (3.19) | 78.62 (3.14) |
| chr19: 3116092-3117059 | GNA11 | intron | Shore | 58.56 (8.13) | 58.2 (8.03) | 72.45 (1.9) |
| chr19: 424120-424779 | SHC2 | intron | Shore | 61.11 (6.47) | 56.3 (9.61) | 72.99 (2.64) |
| chr20: 60513256-60514245 | CDH4 | 3′ UTR | Shore | 58.09 (2.88) | 56.83 (3.9) | 68.25 (3.55) |
| chr22: 43089505-43089920 | A4GALT | CDS | CpG island | 21.23 (1.21) | 21.86 (1.97) | 42.23 (6.59) |
| chr22: 39635092-39635646 | PDGFB | intron | Shelf | 35.88 (3.99) | 34.96 (3) | 45.69 (3.65) |
| chr22: 38168828-38168948 | TRIOBP | intron | open sea | 35.11 (9.23) | 35.92 (5.67) | 52.32 (4.03) |
| chr7: 6437984-6438528 | RAC1 | CDS | Shelf | 65 (3.68) | 65.64 (4.34) | 72.53 (3.8) |
| Chr7: 150972294-150972558 | SMARCD3 | 5′ UTR | Shore | 37.88 (12.75) | 41.32 (10.32) | 54.34 (9.81) |
| Chr16: 1128277-1128918 | SSTR5 | 5′ UTR | CpG island | 41.68 (4.7) | 43.44 (2.89) | 56.84 (11.35) |
| Chr3: 50272811-50273341 | GNAI2 | Promoter | CpG island | 44.72 (2.14) | 45.44 (2.83) | 55.41 (12.57) |
| Chr10: 134899104-134899639 | GPR123 | Promoter | Shore | 60.4 (2.99) | 61.75 (4.97) | 69.48 (6.48) |
| Chr11: 1890055-1890844 | LSP1 | 5′ UTR | Shore | 54.33 (6.38) | 56.56 (3.36) | 64.13 (6.49) |
| Chr19: 5297963-5298605 | PTPRS | intron | Shelf | 59.7 (3.78) | 61.47 (2.56) | 72.57 (7.09) |
| Chr17: 44872642-44872984 | WNT3 | intron | Open Sea | 48.83 (4.17) | 50.08 (4.65) | 64.46 (13.77) |
| Chr20: 62175639-62175878 | SRMS | CDS | Shelf | 43.62 (4.4) | 50.31 (3.39) | 60.39 (8.98) |
| Chr7: 219525-220144 | FAM20C | intron | Shore | 40.32 (2.92) | 42.43 (4) | 53.45 (14.16) |
| Chr3: 13678470-13679044 | FBLN2 | intron | Shore | 55.61 (4.12) | 55.7 (1.79) | 67.16 (10.14) |
| chr6: 27115790-27115898 | HIST1H2BK | Promoter | Shore | 53.61 (5.08) | 53.5 (5.47) | 65.77 (8.31) |
| Chr20: 61463372-61463768 | COL9A3 | CDS | Shelf | 39.27 (4.49) | 37.71 (3.75) | 47.86 (8.67) |
| Chr1: 228136943-228137043 | WNT9A | Promoter | Shore | 49.02 (5.62) | 50.74 (4.35) | 59.12 (16.03) |
| Chr3: 113001460-113001983 | BOC | intron | Open sea | 69.08 (2.21) | 70.45 (2.33) | 75.25 (12.91) |
| Chr7: 298072-298470 | FAM20C | intron | Shore | 49.45 (9.04) | 56.78 (9.02) | 33.25 (19.65) |
| Chr19: 2664459-2664817 | GNG7 | intron | Open sea | 64.27 (3.81) | 62.11 (6.71) | 71.17 (6.8) |
| Chr2: 219489555-219489692 | PLCD4 | Intron | Open sea | 59.54 (4.65) | 64.65 (7.67) | 68.63 (5.95) |
| Chr7: 73456778-73457687 | ELN | CDS | Open sea | 50.75 (4.3) | 51.72 (4.48) | 60.13 (14.97) |
| Chr11: 1033799-1034078 | MUC6 | Intron | Shore | 62.32 (1.99) | 63.14 (2.44) | 68.52 (9.54) |
| Chr5: 495153-495249 | SLC9A3 | Intron | CpG island | 79.68 (3.46) | 83.4 (7.18) | 85.9 (8.69) |
| Chr2: 239030927-239031207 | ESPNL | Intron | Open sea | 54.83 (5.72) | 59.45 (5.22) | 65.2 (9.58) |
| Chr1: 152009695-152010284 | S100A11 | Promoter | Shore | 51.47 (4.63) | 47.05 (12.54) | 58.08 (13.34) |

| | AUC of CRC vs. CTRL | AUC of LS vs. ES |
|---|---|---|
| DMR Location | (95% CI) | (95 % CI) |
| Hypermethylated | | |
| chr3: 129276017-129277607 | 1 (95% CI: 1, 1) | 0.717 (95% CI: 0, 434, 0.954) |
| chr4: 1800772-1801634 | 0.995 (95% CI: 0, 968.1) | 0.626 (95% CI: 0, 337, 0.856) |
| chr4: 1219063-1219488 | 0.99 (95% CI: 0.953.1) | 0.566 (95% CI: 0.303.0.838) |
| chr4: 160256911-160257058 | 1 (95% CI: 1.1) | 0.515 (95% CI: 0.242.0.81) |
| chr8: 134279628-134280406 | 1 (95% CI: 1.1) | 0.616 (95% CI: 0.354.0.852) |
| chr9: 117063843-117064728 | 0.985 (95% CI: 0.94, 1) | 0.788 (95% CI: 0.522, 1) |
| chr9: 139411694-139412139 | 0.99 (95% CI: 0.958, 1) | 0.626 (95% CI: 0.326, 0.883) |
| chr9: 136003298-136003875 | 1 (95% CI: 1, 1) | 0.606 (95% CI: 0.36, 0.842) |
| chr9: 137324620-137325166 | 1 (95% CI: 1, 1) | 0.717 (95% CI: 0.459, 0.919) |
| chr9: 127831756-127831995 | 0.995 (95% CI: 0.972, 1) | 0.475 (95% CI: 0.188, 0.739) |
| chr9: 126123252-126123504 | 1 (95% CI: 1, 1) | 0.576 (95% CI: 0.326, 0.844) |
| chr11: 128839732-128840325 | 1 (95% CI: 1, 1) | 0.515 (95% CI: 0.234, 0.799) |
| chr17: 16329581-16329742 | 0.995 (95% CI: 0.969, 1) | 0.545 (95% CI: 0.287, 0.815) |
| chr17: 78836096-78836514 | 0.995 (95% CI: 0.973, 1) | 0.657 (95% CI: 0.363, 0.917) |
| chr17: 5207615-5207733 | 0.98 (95% CI: 0.932, 1) | 0.606 (95% CI: 0.324, 0.869) |
| chr19: 48981419-48981798 | 1 (95% CI: 1, 1) | 0.596 (95% CI: 0.316, 0.839) |
| chr19: 38673435-38674132 | 0.995 (95% CI: 0.972, 1) | 0.525 (95% CI: 0.22, 0.777) |
| chr19: 4101341-4101586 | 0.985 (95% CI: 0.943, 1) | 0.606 (95% CI: 0.342, 0.857) |
| chr20: 57464965-57465494 | 0.995 (95% CI: 0.964, 1) | 0.667 (95% CI: 0.409, 0.899) |
| chr20: 60737881-60738663 | 1 (95% CI: 1, 1) | 0.778 (95% CI: 0.531, 0.96) |
| Chr19: 58629653-58629967 | 0.962 (95% CI: 0.876, 1) | 0.455 (95% CI: 0.178, 0.694) |
| Chr22: 29075690-29076384 | 0.954 (95% CI: 0.872, 1) | 0.576 (95% CI: 0.316, 0.857) |
| Chr7: 127881049-127881369 | 0.91 (95% CI: 0.795, 0.988) | 0.667 (95% CI: 0.389, 0.9) |
| Chr2: 131673573-131674131 | 0.923 (95% CI: 0.826, 0.993) | 0.727 (95% CI: 0.451, 0.956) |
| Chr5: 140792683-140793474 | 0.93 (95% CI: 0.819, 1) | 0.727 (95% CI: 0.482, 0.961) |
| Chr6: 42695094-42695626 | 0.995 (95% CI: 0.973, 1) | 0.586 (95% CI: 0.266, 0.87) |
| Chr19: 39398053-39398350 | 0.88 (95% CI: 0.755, 0.98) | 0.515 (95% CI: 0.192, 0.865) |
| Chr6: 144329245-144329549 | 0.849 (95% CI: 0.661, 0.993) | 0.656 (95% CI: 0.381, 0.886) |
| Chr12: 54331986-54333458 | 0.847 (95% CI: 0.67, 0.96) | 0.687 (95% CI: 0.429, 0.917) |
| Chr19: 47152581-47152993 | 0.93 (95% CI: 0.826, 1) | 0.505 (95% CI: 0.256, 0.781) |
| Chr7: 1028328-1028614 | 0.93 (95% CI: 0.833, 0.991) | 0.616 (95% CI: 0.353, 0.88) |
| Chr7: 155595151-155596289 | 0.887 (95% CI: 0.74, 0.99) | 0.586 (95% CI: 0.298, 0.829) |
| Chr8: 37823708-37823868 | 0.893 (95% CI: 0.766, 0.993) | 0.667 (95% CI: 0.409, 0.889) |
| Chr19: 49936977-49937165 | 0.914 (95% CI: 0.777, 1) | 0.515 (95% CI: 0.241, 0.754) |
| Chr15: 23931943-23932081 | 0.864 (95% CI: 0.691, 0.992) | 0.687 (95% CI: 0.422, 0.932) |
| Chr7: 2691893-2692388 | 0.857 (95% CI: 0.643, 1) | 0.586 (95% CI: 0.308, 0.859) |
| Chr11: 32452200-32453076 | 0.867 (95% CI: 0.697, 0.982) | 0.404 (95% CI: 0.154, 0.674) |
| Chr2: 19553173-19553431 | 0.854 (95% CI: 0.692, 0.98) | 0.525 (95% CI: 0.254, 0.814) |
| Chr20: 2730191-2730750 | 0.857 (95% CI: 0.661, 0.992) | 0.545 (95% CI: 0.242, 0.826) |
| Chr10: 125425607-125426325 | 0.837 (95% CI: 0.674, 0.954) | 0.707 (95% CI: 0.423, 0.94) |
| Chr4: 41747769-41748363 | 0.843 (95% CI: 0.681, 0.97) | 0.667 (95% CI: 0.379, 0.914) |
| Chr14: 100126403-100126679 | 0.82 (95% CI: 0.66, 0.942) | 0.556 (95% CI: 0.277, 0.809) |
| Chr5: 624972-625147 | 0.831 (95% CI: 0.637, 0.975) | 0.414 (95% CI: 0.141, 0.68) |
| Chr16: 89690046-89690262 | 0.812 (95% CI: 0.654, 0.941) | 0.505 (95% CI: 0.239, 0.795) |
| Chr19: 3452488-3452664 | 0.782 (95% CI: 0.583, 0.928) | 0.909 (95% CI: 0.744, 1) |
| Chr2: 220283249-220283951 | 0.957 (95% CI: 0.885, 1) | 0.586 (95% CI: 0.276, 0.878) |
| Hypomethylated | | |
| chr3: 48677529-48678331 | 1 (95% CI: 1, 1) | 0.515 (95% CI: 0.23, 0.78) |
| chr5: 179695549-179695862 | 1 (95% CI: 1, 1) | 0.884 (95% CI: 0.675, 1) |
| chr7: 98984785-98986056 | 0.955 (95% CI: 0.861, 1) | 0.798 (95% CI: 0.55, 0.995) |
| chr7: 73520260-73520947 | 0.99 (95% CI: 0.955, 1) | 0.636 (95% CI: 0.384, 0.893) |
| chr7: 4208636-4208932 | 0.99 (95% CI: 0.952, 1) | 0.646 (95% CI: 0.375, 0.888) |
| chr8: 18827759-18827909 | 1 (95% CI: 1, 1) | 0.505 (95% CI: 0.23, 0.795) |
| chr9: 136669856-136671417 | 1 (95% CI: 1, 1) | 0.616 (95% CI: 0.283, 0.903) |
| chr9: 141013037-141013769 | 0.99 (95% CI: 0.953, 1) | 0.707 (95% CI: 0.4, 0.939) |
| chr11: 72404012-72404706 | 0.935 (95% CI: 0.834, 1) | 0.848 (95% CI: 0.628, 1) |
| chr12: 52447977-52448157 | 1 (95% CI: 1, 1) | 0.677 (95% CI: 0.437, 0.92) |
| chr12: 57590785-57591112 | 0.96 (95% CI: 0.874, 1) | 0.859 (95% CI: 0.612, 1) |
| chr12: 125296703-125297291 | 0.995 (95% CI: 0.971, 1) | 0.475 (95% CI: 0.21, 0.759) |
| chr13: 111117540-111117853 | 0.937 (95% CI: 0.811, 1) | 0.807 (95% CI: 0.571, 0.987) |
| chr13: 114823604-114823726 | 0.99 (95% CI: 0.959, 1) | 0.646 (95% CI: 0.392, 0.893) |
| chr14: 105239801-105240179 | 0.995 (95% CI: 0.972, 1) | 0.657 (95% CI: 0.379, 0.887) |
| chr14: 104612850-104614023 | 0.92 (95% CI: 0.785, 1) | 0.768 (95% CI: 0.537, 0.964) |
| chr14: 105831329-105832002 | 1 (95% CI: 1, 1) | 0.636 (95% CI: 0.326, 0.895) |
| chr17: 958859-960394 | 1 (95% CI: 1, 1) | 0.556 (95% CI: 0.265, 0.798) |
| chr17: 36638843-36639223 | 1 (95% CI: 1, 1) | 0.636 (95% CI: 0.34, 0.88) |
| chr17: 79074960-79075309 | 1 (95% CI: 1, 1) | 0.616 (95% CI: 0.323, 0.87) |
| chr19: 42492254-42492761 | 0.98 (95% CI: 0.926, 1) | 0.424 (95% CI: 0.158, 0.695) |
| chr19: 3116092-3117059 | 0.995 (95% CI: 0.964, 1) | 0.545 (95% CI: 0.241, 0.846) |
| chr19: 424120-424779 | 0.995 (95% CI: 0.967, 1) | 0.707 (95% CI: 0.455, 0.945) |
| chr20: 60513256-60514245 | 0.985 (95% CI: 0.94, 1) | 0.616 (95% CI: 0.322, 0.859) |
| chr22: 43089505-43089920 | 1 (95% CI: 1, 1) | 0.545 (95% CI: 0.274, 0.842) |
| chr22: 39635092-39635646 | 0.985 (95% CI: 0.935, 1) | 0.566 (95% CI: 0.297, 0.812) |
| chr22: 38168828-38168948 | 0.985 (95% CI: 0.931, 1) | 0.545 (95% CI: 0.295, 0.803) |
| chr7: 6437984-6438528 | 0.925 (95% CI: 0.815, 1) | 0.475 (95% CI: 0.213, 0.744) |
| Chr7: 150972294-150972558 | 0.911 (95% CI: 0.787, 0.99) | 0.657 (95% CI: 0.381, 0.895) |
| Chr16: 1128277-1128918 | 0.89 (95% CI: 0.718, 1) | 0.636 (95% CI: 0.353, 0.869) |
| Chr3: 50272811-50273341 | 0.908 (95% CI: 0.748, 1) | 0.596 (95% CI: 0.306, 0.872) |
| Chr10: 134899104-134899639 | 0.89 (95% CI: 0.748, 1) | 0.657 (95% CI: 0.403, 0.895) |
| Chr11: 1890055-1890844 | 0.873 (95% CI: 0.755, 0.967) | 0.646 (95% CI: 0.405, 0.885) |
| Chr19: 5297963-5298605 | 0.937 (95% CI: 0.771, 1) | 0.556 (95% CI: 0.265, 0.816) |
| Chr17: 44872642-44872984 | 0.918 (95% CI: 0.809, 0.991) | 0.545 (95% CI: 0.262, 0.816) |
| Chr20: 62175639-62175878 | 0.917 (95% CI: 0.755, 1) | 0.889 (95% CI: 0.691, 1) |
| Chr7: 219525-220144 | 0.868 (95% CI: 0.697, 0.993) | 0.687 (95% CI: 0.403, 0.944) |
| Chr3: 13678470-13679044 | 0.85 (95% CI: 0.683, 0.987) | 0.545 (95% CI: 0.277, 0.78) |
| chr6: 27115790-27115898 | 0.877 (95% CI: 0.69, 0.996) | 0.525 (95% CI: 0.219, 0.839) |
| Chr20: 61463372-61463768 | 0.842 (95% CI: 0.629, 0.974) | 0.606 (95% CI: 0.333, 0.868) |
| Chr1: 228136943-228137043 | 0.825 (95% CI: 0.619, 0.993) | 0.586 (95% CI: 0.333, 0.848) |
| Chr3: 113001460-113001983 | 0.81 (95% CI: 0.619, 0.987) | 0.657 (95% CI: 0.374, 0.872) |
| Chr7: 298072-298470 | 0.807 (95% CI: 0.637, 0.944) | 0.788 (95% CI: 0.558, 0.967) |
| Chr19: 2664459-2664817 | 0.796 (95% CI: 0.619, 0.937) | 0.616 (95% CI: 0.342, 0.91) |
| Chr2: 219489555-219489692 | 0.814 (95% CI: 0.641, 0.962) | 0.707 (95% CI: 0.415, 0.91) |
| Chr7: 73456778-73457687 | 0.783 (95% CI: 0.578, 0.956) | 0.545 (95% CI: 0.267, 0.79) |
| Chr11: 1033799-1034078 | 0.783 (95% CI: 0.547, 0.956) | 0.616 (95% CI: 0.295, 0.858) |
| Chr5: 495153-495249 | 0.809 (95% CI: 0.589, 0.995) | 0.667 (95% CI: 0.396, 0.925) |
| Chr2: 239030927-239031207 | 0.8 (95% CI: 0.587, 0.972) | 0.768 (95% CI: 0.517, 0.948) |
| Chr1: 152009695-152010284 | 0.75 (95% CI: 0.551, 0.903) | 0.606 (95% CI: 0.316, 0.89) |

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A gene panel comprising a at least two differentially methylated genes selected from PLXND1, FGFR3, CTBP1, RAPGEF2, NDRG1, COL27A1, NOTCH1, RALGDS, RXRA, SCA1, CRB2, ARHGAP32, TRPV2, RPTOR, RABEP1, CYTH2, SIPAIL3, MAP2K2, GNAS, SS18L1, ZSCAN18, TTC28, LEP, ARHGEF4, PCDHGA10, ATP6VOCP3, NFKBIB, PLAGL1, HOXC13, DACT3, CYP2W1, SHH, ADRB3, SLC17A7, NDN, TTYH3, WT1, OSR1, EBF4, GPR26, PHOX2B, HHIPL1, CEP72, DPEP1, NFIC, DES, SDHAF1, RXRA, CELSR3, MAPK9, ARPC1B, LIMK1, SDK1, PSD3, VAV2, CACNA1B, ARAP1, NR4A1, LRP1, SCARB1, COL4A2, RASA3, AKT1, KIF26A, PACS2, ABR, ARHGAP23, BAIAP2, ATP1A3, GNA11, SHC2, CDH4, A4GALT, PDGFB, TRIOBP, RAC1, SMARCD3, SSTR5, GNAI2, GPR123, LSP1, PTPRS, WNT3, SRMS, FAM20C, FBLN2, HIST1H2BK, COL9A3, WNT9A, BOC, FAM20C, GNG7, PLCD4, ELN, MUC6, SLC9A3, ESPNL and S100A11.
